(19) United States
(12) Patent Application Publication
(10) Pub. No.: US 2026/0083497 A1
(43) Pub. Date: Mar. 26, 2026

(54) SYSTEM FOR ABLATION ZONE PREDICTION

(71) Applicant: Covidien LP, Mansfield, MA (US)

(72) Inventor: William J. DICKHANS, Lafayette, CO (US)

(21) Appl. No.: 19/112,641

(22) PCT Filed: Oct. 2, 2023
(86) PCT No.: PCT/US2023/075670

§ 371 Date: Mar. 18, 2025

Related U.S. Application Data
(60) Provisional application No. 63/412,731, filed on Oct. 3, 2022.

(51) Int. Cl. A61B 18/14; A61B 18/00; A61B 34/10

(52) U.S. Cl. CPC A61B 18/1492; A61B 34/10; A61B 2018/00541; A61B 2018/00577; A61B 2018/00982; A61B 2034/104; A61B 2034/105; A61B 2034/107

(57) ABSTRACT

An ablation system includes a computing device and an ablation device configured to ablate a target. The computing device is configured to generate a three dimensional model based on functional respiratory imaging data of a patient and to predict an ablation zone based on the functional respiratory imaging data.

### CROSS-REFERENCE TO RELATED APPLICATIONS

This application claims the benefit of the filing date of provisional U.S. Patent Application No. 63/412,731, filed on Oct. 3, 2022.

### FIELD

The present disclosure relates to systems, methods, and devices for predicting ablation zones in a microwave ablation treatment procedure.

### BACKGROUND

When planning a treatment procedure, clinicians often rely on patient data including X-ray data, computed tomography (CT) scan data, magnetic resonance imaging (MRI) data, or other imaging data that allows the clinician to view the internal anatomy of a patient. The clinician utilizes the patient data to identify targets of interest and to develop strategies for accessing the targets of interest for the treatment procedure.

Existing imaging modalities introduce unique challenges when monitoring the progression of an ablation zone during an ablation procedure. Ultrasound imaging displays “bubbles” or ground glass opacity as energy is delivered, making it difficult to determine the edge of the actual ablation zone during progression of the application of energy. CT imaging provides “near-time” imaging that reflects the ablation zone size at the time that the last scan was captured, however, does not reflect ablation growth as it occurs in real time.

Additionally, predicting the outcome of an ablation procedure (e.g., the final ablation zone characteristics) is difficult as one standard data set used to predict the outcome of an ablation procedure may not be generic to multiple patients and/or multiple targets.

### SUMMARY

Systems and methods for predicting ablation zones in a microwave ablation treatment procedure are provided.

According to an aspect of the present disclosure, an ablation system includes an ablation device configured to ablate a target, and a computing device. The computing device is configured to receive functional respiratory imaging data of a patient and generate a three dimensional model of the lung of the patient based on the received functional respiratory imaging data of the patient. The computing device is further configured to predict lung movement based on the received functional respiratory imaging data of the patient and predict movement of the ablation device relative to the lung and the target during a predetermined duration of ablation. The computing device predicts an ablation zone position and margin relative to the target based on the received functional respiratory imaging data of the patient and the predicted movement of the ablation device relative to the lung and the target during the predetermined duration of ablation.

In an aspect, the computing device is configured to calculate a best path for the ablation device to approach the target that would create a maximum ablation margin based on the received functional respiratory imaging data of the patient. The calculated best path may include a path through a luminal network and a point on an airway wall to puncture through for placement of the ablation device outside of the luminal network.

In an aspect, the computing device is configured to calculate a best position for final placement of the ablation device relative to the target that would create a maximum ablation margin based on the received functional respiratory imaging data of the patient. The calculated best position may be outside of an airway of the lung.

In an aspect, the computing device is configured to receive post-procedure data corresponding to an actual ablation zone position and margin and compare the post-procedural data corresponding to the actual ablation zone position and margin with the predicted ablation zone position and margin to calculate a deviation between the actual ablation zone position and margin with the predicted ablation zone position and margin. The computing device may be configured to execute a learning algorithm to learn from the calculated deviation for other predictions of ablation zone positions and margins.

In an aspect, the functional respiratory imaging data may include data corresponding to at least one of blood vessel volume, airway volume, lung volume, airway resistance, internal airflow distribution, ventilation, perfusion, air trapping, aerosol deposition, fissure integrity, fibrosis, or emphysema.

In accordance with another aspect of the disclosure, an ablation system includes a computing device. The computing device is configured to receive functional respiratory imaging data of a lung of a patient and a target within the lung, predict lung movement based on the received functional respiratory imaging data of the patient, predict movement of an ablation device relative to the lung and the target during a predetermined duration of ablation, and predict an ablation zone position and margin relative to the target based on the received functional respiratory imaging data of the patient and the predicted movement of the ablation device relative to the lung and the target during the predetermined duration of ablation.

In an aspect, the computing device is configured to generate a three dimensional model of the lung of the patient based on the received functional respiratory imaging data of the patient.

In an aspect, the computing device may be configured to calculate a best path for the ablation device to approach the target that would create a maximum ablation margin based on the received functional respiratory imaging data of the patient. The calculated best path may include a path through a luminal network and a point on an airway wall to puncture through for placement of the ablation device outside of the luminal network.

In an aspect, the computing device is configured to calculate a best position for final placement of the ablation device relative to the target that would create a maximum ablation margin based on the received functional respiratory imaging data of the patient. The calculated best position may be outside of an airway of the lung.

In an aspect, the computing device is configured to receive post-procedure data corresponding to an actual ablation zone position and margin and compare the post-procedural data corresponding to the actual ablation zone position and margin with the predicted ablation zone position and margin to calculate a deviation between the actual ablation zone position and margin with the predicted ablation zone position and margin. The computing device may be configured to execute a learning algorithm to learn from the calculated deviation for other predictions of ablation zone positions and margins.

In an aspect, the functional respiratory imaging data may include data corresponding to at least one of blood vessel volume, airway volume, lung volume, airway resistance, internal airflow distribution, ventilation, perfusion, air trapping, aerosol deposition, fissure integrity, fibrosis, or emphysema.

In accordance with another aspect of the disclosure, an ablation system includes a computing device. The computing device is configured to receive functional respiratory imaging data of a lung of a patient and a target within the lung, predict an ablation zone position and margin relative to the target based on the received functional respiratory imaging data of the patient, receive post-procedure data corresponding to an actual ablation zone position and margin, and compare the post-procedural data corresponding to the actual ablation zone position and margin with the predicted ablation zone position and margin to calculate a deviation between the actual ablation zone position and margin with the predicted ablation zone position and margin.

In an aspect, the computing device is configured to execute a learning algorithm to learn from the calculated deviation for other predictions of ablation zone positions and margins.

In an aspect, the functional respiratory imaging data may include data corresponding to at least one of blood vessel volume, airway volume, lung volume, airway resistance, internal airflow distribution, ventilation, perfusion, air trapping, aerosol deposition, fissure integrity, fibrosis, or emphysema.

Any of the above aspects and embodiments of the present disclosure may be combined without departing from the scope of the present disclosure.

### BRIEF DESCRIPTION OF THE DRAWINGS

Objects and features of the presently disclosed system and method will become apparent to those of ordinary skill in the art when descriptions of various embodiments thereof are read with reference to the accompanying drawings, of which:

FIG. 1 is a schematic diagram of a microwave ablation procedure system in accordance with an illustrative aspect of the present disclosure;

FIG. 2 is a schematic diagram of a computing device which forms part of the microwave ablation procedure system of FIG. 1 in accordance with an aspect of the present disclosure;

FIG. 3 illustrates an example user interface displaying a predicted ablation zone in accordance with an aspect of the present disclosure; and

FIG. 4 is flowchart illustrating a method for predicting an ablation zone in accordance with an aspect of the present disclosure.

### DETAILED DESCRIPTION

The present disclosure provides a system and method for predicting an ablation zone during a microwave ablation treatment procedure using functional respiratory imaging data. Existing lung ablation systems rely on a fixed look up table to determine the power and time settings needed to create an ablation. This can lead to variability as the lung has vasculature along with air and fluid-filled sacs. Tumors present additional variability depending on the morphology. An ablation zone can turn out to be much smaller than expected when confirmed during or after a procedure on a confirmation scan causing the physician to change plans intraprocedurally, adding time and frustration to the procedure, or causing the physician to determine that another procedure is required in order to completely ablate the target.

The present disclosure describes a system and method that utilizes multiple data sets (e.g., multiple lung volume scans during different respiratory stages) to understand how the lung moves in 3D space along with other parameters obtained via functional respiratory imaging. Functional respiratory imaging may provide insight into data factors such as blood vessel volume, airway volume, lung volume, airway resistance, internal airflow distribution, ventilation/perfusion, air trapping, aerosol deposition, fissure integrity, fibrosis and/or emphysema. With this information, the ablation system factors how the lung is moving, and the parameters available via functional respiratory imaging are inputs into a prediction model for determining how and where to place the ablation device (e.g., a flexible ablation catheter) to create a maximum ablation margin. This model also factors a prediction of the ablation catheter's movement relative to the target and other lung structures that may impact the ablation zone during an ablation procedure.

Although the present disclosure will be described in terms of specific illustrative embodiments, it will be readily apparent to those skilled in the art that various modifications, rearrangements, and substitutions may be made without departing from the spirit of the present disclosure. The scope of the present disclosure is defined by the claims appended hereto.

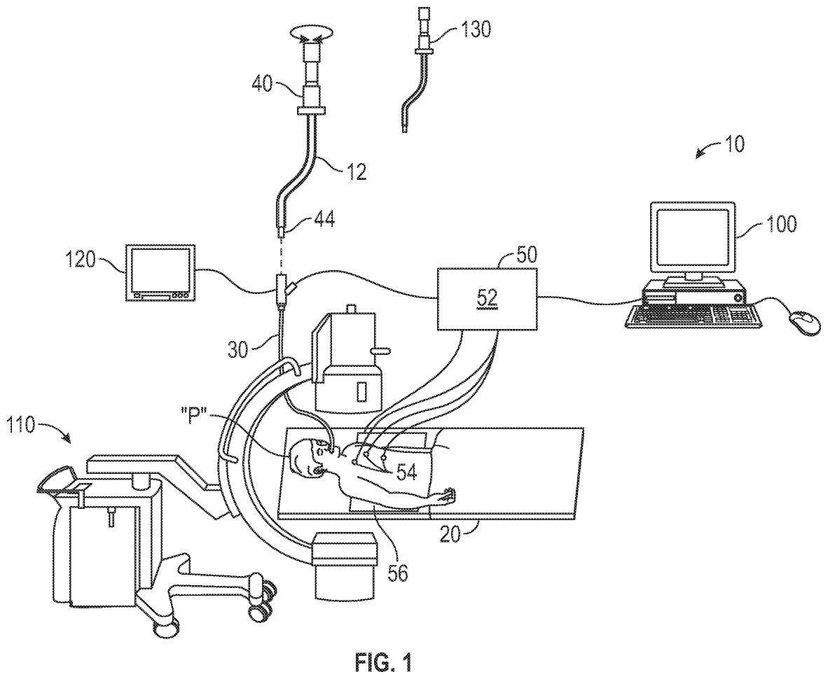

FIG. 1 depicts an Electromagnetic Navigation (EMN) system 10 configured for reviewing CT image data to identify one or more targets, planning a pathway to an identified target (planning phase), navigating an extended working channel (EWC) 12 of a catheter guide assembly 40 to a target (navigation phase) via a user interface, and confirming placement of the EWC 12 relative to the target. One such EMN system is the ELECTROMAGNETIC NAVIGATION BRONCHOSCOPY® system currently sold by Medtronic PLC. The target may be tissue of interest identified by review of the CT image data during the planning phase. Following navigation, a medical instrument, such as a biopsy tool or other tool, may be inserted into the EWC 12 to obtain a tissue sample (or perform a treatment) from the tissue located at, or proximate to, the target.

As shown in FIG. 1, EWC 12 is part of a catheter guide assembly 40. In practice, the EWC 12 is inserted into bronchoscope 30 for access to a luminal network of the patient “P.” Specifically, EWC 12 of catheter guide assembly 40 may be inserted into a working channel of bronchoscope 30 for navigation through a patient's luminal network. A distal portion of the EWC 12 includes a sensor 44. The position and orientation of the sensor 44 relative to the reference coordinate system, and thus the distal portion of the EWC 12, within an electromagnetic field can be derived. Catheter guide assemblies 40 are currently marketed and sold by Medtronic PLC under the brand names SUPERDIMENSION® Procedure Kits, or EDGE™ Procedure Kits, and are contemplated as useable with the disclosure. System 10 and its components are described in greater detail below.

EMN system 10 generally includes an operating table 20 configured to support a patient “P;” a bronchoscope 30 configured for insertion through the patient's “P's” mouth into the patient's “P's” airways; monitoring equipment 120 coupled to bronchoscope 30 (e.g., a video display for displaying the video images received from the video imaging system of bronchoscope 30); a tracking system 50 including a tracking module 52, a plurality of reference sensors 54 and a transmitter mat 56; and a computing device 100 including software and/or hardware used to facilitate identification of a target, pathway planning to the target, navigation of a medical instrument to the target, and confirmation of placement of EWC 12 and/or placement of a suitable device through EWC 12 and relative to a target.

A fluoroscopic imaging device 110 capable of acquiring fluoroscopic or x-ray images or video of the patient “P” is also included in this particular aspect of system 10. The fluoroscopic data (e.g., images, series of images, or video) captured by the fluoroscopic imaging device 110 may be stored within the fluoroscopic imaging device 110 or transmitted to computing device 100 for storage, processing, and display. Additionally, the fluoroscopic imaging device 110 may move relative to the patient “P” so that images may be acquired from different angles or perspectives relative to the patient “P” to create a fluoroscopic video from a fluoroscopic sweep. Fluoroscopic imaging device 110 may include a single imaging device or more than one imaging device. In embodiments including multiple imaging devices, each imaging device may be a different type of imaging device or the same type.

Computing device 100 may be any suitable computing device including a processor and storage medium, wherein the processor is capable of executing instructions stored on the storage medium. The computing device 100 may further include a database configured to store patient data, CT data sets including CT images, fluoroscopic data sets including fluoroscopic images and video, navigation plans, and any other such data. Although not explicitly illustrated, the computing device 100 may include inputs, or may otherwise be configured to receive, CT data sets, fluoroscopic images/video and other data described herein. Additionally, computing device 100 includes a display configured to display graphical user interfaces.

With respect to the planning phase, computing device 100 utilizes previously acquired CT image data for generating and viewing a three dimensional model of the patient's “P's” airways, enables the identification of a target on the three dimensional model (automatically, semi-automatically, or manually), and allows for determining a pathway through the patient's “P's” airways to tissue located at and around the target. More specifically, CT images acquired from previous CT scans are processed and assembled into a three dimensional CT volume, which is then utilized to generate a three dimensional model of the patient's “P's” airways. The three dimensional model may be displayed on a display associated with computing device 100, or in any other suitable fashion. Using computing device 100, various views of the three dimensional model or enhanced two dimensional images generated from the three dimensional model are presented. The enhanced two dimensional images may possess some three dimensional capabilities because they are generated from three dimensional data. The three dimensional model may be manipulated to facilitate identification of target on the three dimensional model or two dimensional images, and selection of a suitable pathway (e.g., route to be following during navigation) through the patient's “P's” airways to access tissue located at the target can be made. Once selected, the pathway plan, three dimensional model, and images derived therefrom, can be saved and exported to a navigation system for use during the navigation phase(s).

With respect to the navigation phase, a six degrees-of-freedom electromagnetic tracking system 50 may be utilized for performing registration of the images and the pathway for navigation, although other configurations are also contemplated. Tracking system 50 includes a tracking module 52, a plurality of reference sensors 54, and a transmitter mat 56. Tracking system 50 is configured for use with a sensor 44 of catheter guide assembly 40 to track the electromagnetic position thereof within an electromagnetic coordinate system.

Transmitter mat 56 is positioned beneath patient “P.” Transmitter mat 56 generates an electromagnetic field around at least a portion of the patient “P” within which the position of a plurality of reference sensors 54 and the sensor element 44 can be determined with use of a tracking module 52. One or more of reference sensors 54 are attached to the chest of the patient “P.” The six degrees of freedom coordinates of reference sensors 54 are sent to computing device 100 (which includes the appropriate software) where they are used to calculate a patient coordinate frame of reference. Registration, as detailed below, is generally performed to coordinate locations of the three dimensional model and two dimensional images from the planning phase with the patient's “P's” airways as observed through the bronchoscope 30, and allow for the navigation phase to be undertaken with precise knowledge of the location of the sensor 44, even in portions of the airway where the bronchoscope 30 cannot reach.

Registration of the patient's “P's” location on the transmitter mat 56 is performed by moving sensor 44 through the airways of the patient's “P.” More specifically, data pertaining to locations of sensor 44, while EWC 12 is moving through the airways, is recorded using transmitter mat 56, reference sensors 54, and tracking module 52. A shape resulting from this location data is compared to an interior geometry of passages of the three dimensional model generated in the planning phase, and a location correlation between the shape and the three dimensional model based on the comparison is determined, e.g., utilizing the software on computing device 100. In addition, the software identifies non-tissue space (e.g., air filled cavities) in the three dimensional model. The software aligns, or registers, an image representing a location of sensor 44 with a the three dimensional model and two dimensional images generated from the three dimension model, which are based on the recorded location data and an assumption that sensor 44 remains located in non-tissue space in the patient's “P's” airways. Alternatively, a manual registration technique may be employed by navigating the bronchoscope 30 with the sensor 44 to pre-specified locations in the lungs of the patient “P”, and manually correlating the images from the bronchoscope to the model data of the three dimensional model.

Following registration of the patient “P” to the image data and pathway plan, a user interface is displayed in the navigation software which sets forth the pathway that the clinician is to follow to reach the target.

Once EWC 12 has been successfully navigated proximate the target as depicted on the user interface, the EWC 12 is in place as a guide channel for guiding medical instruments including without limitation, optical systems, ultrasound probes, marker placement tools, biopsy tools, ablation tools (i.e., microwave ablation devices), laser probes, cryogenic probes, sensor probes, and aspirating needles to the target. In an aspect, ablation device 130 is guided through EWC 12 for placement relative to a target and ablation of the target.

Ablation device 130 is a surgical instrument having a microwave ablation antenna that is used to ablate tissue, such as a lesion or tumor, (hereinafter referred to as a “target”) by using electromagnetic radiation or microwave energy to heat tissue in order to denature or kill cancerous cells. Ablation device 130 may be a rigid surgical instrument configured for percutaneous insertion and navigation to a target or may be a flexible catheter configured to be navigated to a target via a patient's luminal network.

In addition to the EM tracking system, the surgical instruments at targets may also be visualized by using another imaging system (e.g., ultrasound imaging, fluoroscopic imaging, computed tomography imaging, etc.). For example, an ultrasound imaging device, such as an ultrasound wand, may be used to image the patient's body during the microwave ablation procedure to visualize the location of the surgical instruments, such as ablation device 130, inside the patient's body. In an aspect, fluoroscopic imaging device 110 is utilized for imaging of the target following navigation of the ablation device 130 or during navigation of the ablation device 130 to the target.

The location of ablation device 130 within the body of the patient may be tracked during the surgical procedure. An example method of tracking the location of ablation device 130 is by using the EM tracking system, which tracks the location of ablation device 130 by tracking sensors attached to or incorporated in ablation device 130 or other devices utilized to aid in navigation of the ablation device 130 to the target.

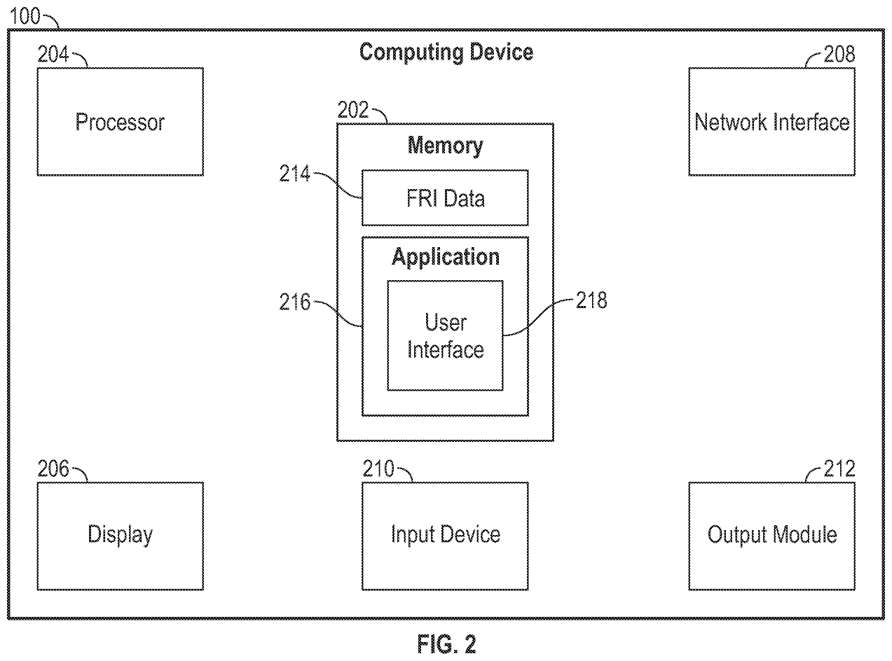

FIG. 2 illustrates a system diagram of computing device 100. Computing device 100 may include memory 202, processor 204, display 206, network interface 208, input device 210, and/or output module 212. Memory 202 includes any non-transitory computer-readable storage media for storing data and/or software that is executable by processor 204 and which controls the operation of computing device 100. In an embodiment, memory 202 may include one or more solid-state storage devices such as flash memory chips. Alternatively or in addition to the one or more solid-state storage devices, memory 202 may include one or more mass storage devices connected to the processor 204 through a mass storage controller (not shown) and a communications bus (not shown). Although the description of computer-readable media contained herein refers to a solid-state storage, it should be appreciated by those skilled in the art that computer-readable storage media can be any available media that can be accessed by the processor 204. That is, computer readable storage media includes non-transitory, volatile and non-volatile, removable and non-removable media implemented in any method or technology for storage of information such as computer-readable instructions, data structures, program modules, or other data. For example, computer-readable storage media includes RAM, ROM, EPROM, EEPROM, flash memory or other solid state memory technology, CD-ROM, DVD, Blu-Ray or other optical storage, magnetic cassettes, magnetic tape, magnetic disk storage or other magnetic storage devices, or any other medium which can be used to store the desired information and which can be accessed by computing device 100.

Memory 202 may store application 216 and/or functional respiratory imaging data 214 of one or more patients. Application 216 may, when executed by processor 204, cause display 206 to present user interface 218. Processor 204 may be a general-purpose processor, a specialized graphics processing unit (GPU) configured to perform specific graphics processing tasks while freeing up the general-purpose processor to perform other tasks, and/or any number or combination of such processors. Display 206 may be touch sensitive and/or voice activated, enabling display 206 to serve as both an input and output device. Alternatively, a keyboard (not shown), mouse (not shown), or other data input devices may be employed. Network interface 208 may be configured to connect to a network such as a local area network (LAN) consisting of a wired network and/or a wireless network, a wide area network (WAN), a wireless mobile network, a Bluetooth network, and/or the internet. For example, computing device 100 may receive functional respiratory imaging data, DICOM imaging data, computed tomographic (CT) image data, or other imaging data, of a patient from an imaging workstation 150 and/or a server, for example, a hospital server, internet server, or other similar servers, for use during surgical ablation planning. Patient functional respiratory imaging data may also be provided to computing device 100 via a removable memory 202. Computing device 100 may receive updates to its software, for example, application 216, via network interface 208. Computing device 100 may also display notifications on display 206 that a software update is available.

Input device 210 may be any device by means of which a user may interact with computing device 100, such as, for example, a mouse, keyboard, foot pedal, touch screen, and/or voice interface. Output module 212 may include any connectivity port or bus, such as, for example, parallel ports, serial ports, universal serial busses (USB), or any other similar connectivity port known to those skilled in the art.

Application 216 may be one or more software programs stored in memory 202 and executed by processor 204 of computing device 100. During a planning phase, application 216 guides a clinician through a series of steps to identify a target, size the target, size a treatment zone, and/or determine an access route to the target for later use during the procedure phase. In some embodiments, application 216 is loaded on computing devices in an operating room or other facility where surgical procedures are performed, and is used as a plan or map to guide a clinician performing a surgical procedure, but without any feedback from ablation device 130 used in the procedure to indicate where ablation device 130 is located in relation to the plan. In other embodiments, system 10 provides computing device 100 with data regarding the location of ablation device 130 within the body of the patient, such as by EM tracking, which application 216 may then use to indicate on the plan where ablation device 130 is located.

Application 216 may be installed directly on computing device 100, or may be installed on another computer, for example, a central server, and opened on computing device 100 via network interface 208. Application 216 may run natively on computing device 100, as a web-based application, or any other format known to those skilled in the art. In some embodiments, application 216 will be a single software program having all of the features and functionality described in the present disclosure. In other embodiments, application 216 may be two or more distinct software programs providing various parts of these features and functionality. For example, application 216 may include one software program for use during the planning phase, and a second software program for use during the procedure phase of the microwave ablation treatment. In such instances, the various software programs forming part of application 216 may be enabled to communicate with each other and/or import and export various settings and parameters relating to the microwave ablation treatment and/or the patient to share information. For example, a treatment plan and any of its components generated by one software program during the planning phase may be stored and exported to be used by a second software program during the procedure phase.

Application 216 communicates with a user interface 218 that generates a user interface for presenting visual interactive features to a clinician, for example, on display 206 and for receiving clinician input, for example, via a user input device. For example, user interface 218 may generate a graphical user interface (GUI) and output the GUI to display 206 for viewing by a clinician.

Computing device 100 is linked to display 110, thus enabling computing device 100 to control the output on display 110 along with the output on display 206. Computing device 100 may control display 110 to display output which is the same as or similar to the output displayed on display 206. For example, the output on display 206 may be mirrored on display 110. Alternatively, computing device 100 may control display 110 to display different output from that displayed on display 206. For example, display 110 may be controlled to display guidance images and information during the microwave ablation procedure, while display 206 is controlled to display other output, such as configuration or status information.

As used herein, the term “clinician” refers to any medical professional (i.e., doctor, surgeon, nurse, or the like) or other user of the treatment planning system 10 involved in planning, performing, monitoring, and/or supervising a medical procedure involving the use of the embodiments described herein.

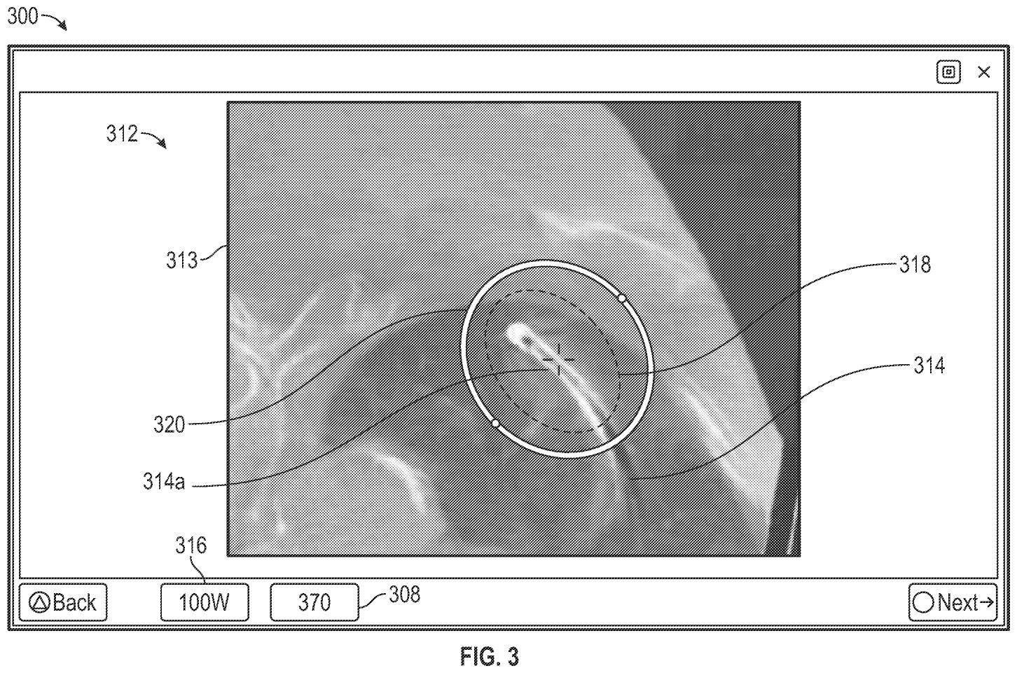

FIG. 3 illustrates an example user interface 300 displayable on one or both of display 110 and display 206 during an ablation procedure. During an ablation procedure, the clinician navigates ablation device 130 (displayed as ablation device 314 on the user interface 300) along a pathway to the target (e.g., utilizing an imaging device such as, for example, a fluoroscopic, CT, or hybrid three dimensional fluoro-CT imaging device, to visualize placement of the ablation device 130 relative to the target). While ablation device 130 is navigated, application 216 tracks the location of ablation device 130 or a catheter through which the ablation device is placed inside the patient's body and displays the tracked location of ablation device 130 or catheter overlayed onto imaging data of the patient (e.g., patient CT data, ultrasound imaging data, fluoroscopic imaging data, hybrid three dimensional fluoro-CT imaging data etc.) on the user interface. In addition, the application 216 may project a vector (not shown) displayable on the user interface 300 extending from the end of the ablation device 130 to give an indication to the clinician of the intersecting tissue along the trajectory of the ablation device 130. In this manner, the clinician can alter the approach to a lesion or tumor to optimize the placement with minimum of trauma or follow a planned pathway to the target.

User interface 300 includes a navigation view 312 including patient images 313 captured during the procedure or captured preoperatively. For example, navigation view 312 may include a three dimensional rendering of the patient's lungs generated from DICOM data. User interface 300 also includes a view for displaying status messages relating to the ablation procedure, such as a power setting 316 of the ablation device 130, duration of the ablation and/or a time remaining until the ablation procedure is complete, progression of the ablation, and/or temperature feedback 308 from a temperature sensor. The navigation view 312 includes a representation 314 of ablation device 130 as well as a shadow indicator 314a representing the portion of ablation device 130 which lies below a displayed two dimensional or three dimensional imaging plane, a simulation of ablation growth 318 showing the progression of the ablation zone during application of energy overlayed onto the patient images 313 of the surgical site, and a total predicted ablation zone 320 showing the area which will be ablated if the ablation procedure is allowed to run to completion. The navigation view 312 may include three dimensional renderings generated from DICOM image data, for example, a three dimensional rendering generated from a CT scan of the patient.

Dimensions of the simulation of ablation growth 318 and the total predicted ablation zone 320 may be based on expected ablation zone sizes at different energy application durations, which is calculated based on functional respiratory imaging data (e.g., of the patient and that of historical functional respiratory imaging data sets). The simulation of ablation growth 318 may include a solid outer edge which increases in size based on the duration of energy application, a jagged line outer edge which moves and increases in size based on the duration of energy application, and/or a pulsing line outer edge which fades between appearing and disappearing and which increases in size based on the duration of energy application and the functional respiratory imaging data. The appearing and disappearing pulsing line of the simulation of ablation growth 318 prevents the simulation of ablation growth 318 from blocking visibility of objects in the displayed patient images 313.

In aspects, dimensions of the simulation of ablation growth 318 and/or total predicted ablation zone 320 may additionally or alternatively be based on one or more other data sets such as previously acquired data samples, MRI telemetry data, and/or inferences from contrast enhanced ultrasound. In aspects, the functional respiratory imaging data, which is used by the computing device 100 to calculate the predicted ablation zone 320 includes data corresponding to at least one of blood vessel volume, airway volume, lung volume, airway resistance, internal airflow distribution, ventilation, perfusion, air trapping, aerosol deposition, fissure integrity, fibrosis, or emphysema.

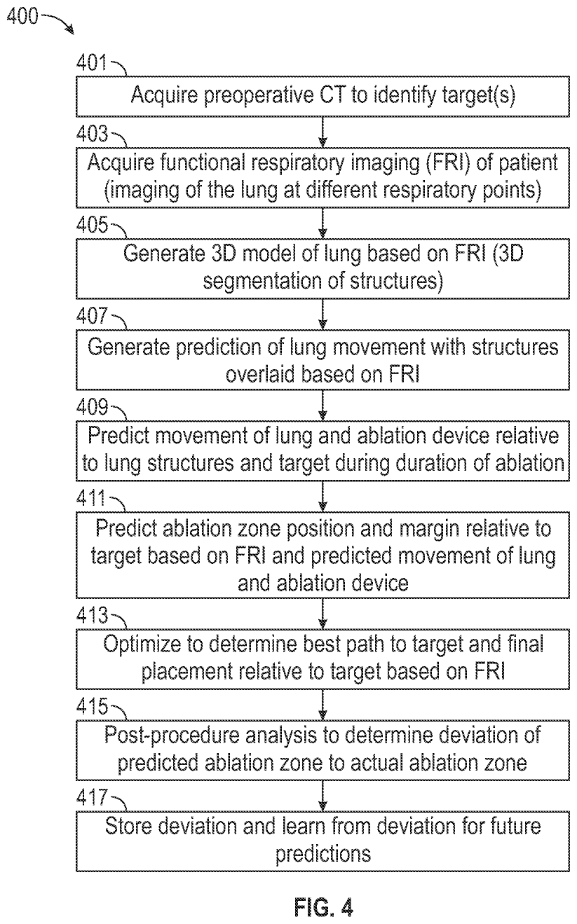

Turning to FIG. 4, a method for predicting an ablation zone is illustrated and described as method 400. Method 400 is described as being executed by computing device 100, but some or all of the steps of method 400 may be implemented by one or more other components of the system 10, alone or in combination. Additionally, although method 400 is illustrated and described as including specific steps, and is described as being carried out in a particular order, it is understood that method 400 may include some or all of the steps described and may be carried out in any order not specifically described.

Method 400 begins at step 401 where computing device 100 acquires preoperative CT data of a patient's lungs to identify one or more targets requiring ablation. In step 403, computing device 100 acquires or otherwise receives functional respiratory imaging data of the patient which includes imaging of the lung at different respiratory points and which provides additional insight into the patient's lungs. For example, the functional respiratory imaging data may include data corresponding to at least one of blood vessel volume, airway volume, lung volume, airway resistance, internal airflow distribution, ventilation, perfusion, air trapping, aerosol deposition, fissure integrity, fibrosis, or emphysema.

In step 405, the computing device 100 generates a 3D model of the patient's lungs based on the functional respiratory imaging data, which may include segmentation of the structures and airways within the lungs. In step 407, the computing device generates a 3D model of movement of the lungs based on the functional respiratory imaging data. The 3D model generated in step 407 may be displayed on a display for analysis.

In step 409, the computing device 100 generates a prediction of movement of the lung and movement of the ablation device 130 relative to structures of the lung and the target during a predetermined duration of an ablation procedure (e.g., a 10 minute ablation procedure). In step 411, the computing device 100 predicts an ablation zone position and margin relative to the target based on the received functional respiratory imaging data of the patient and the predicted movement of the ablation device 130 relative to the lung and the target during the predetermined duration of ablation.

Method 400 may additionally include step 413, where the computing device 100 performs an optimization step to calculate a best path for the ablation device 130 to approach the target that would create a maximum ablation margin based on the received functional respiratory imaging data of the patient. For example, in step 413, the computing device 100 may determine that the best path for the ablation device 130 to approach the target includes a path through a luminal network and a point on an airway wall to puncture through for placement of the ablation device 130 outside of the luminal network. Step 413 may additionally, or alternatively, include calculating a best position for final placement of the ablation device 130 relative to the target that would create a maximum ablation margin based on the received functional respiratory imaging data of the patient. For example, the computing device 100 may determine that the best position for final placement of the ablation device 130 be a position outside of the airway wall for ablation of a target inside or outside of the airway wall, depending on the functional respiratory imaging data.

In step 415, the computing device 100 receives post-procedure data corresponding to an actual ablation zone position and margin and compares the post-procedural data corresponding to the actual ablation zone position and margin with the predicted ablation zone position and margin to calculate a deviation between the actual ablation zone position and margin with the predicted ablation zone position and margin. The post-procedure data may include an image data set, for example CT data, that depicts the outcome of an ablation procedure performed on a target. In step 417, the computing device 100 executes a learning algorithm to learn from the calculated deviation for other predictions of ablation zone positions and margins for the same or other patients. The learning algorithm may utilize artificial intelligence, data models, or machine learning techniques which may include, but are not limited to, neural networks, convolutional neural networks (CNN), recurrent neural networks (RNN), generative adversarial networks (GAN), Bayesian Regression, Naive Bayes, nearest neighbors, least squares, means, and support vector regression, among other data science and artificial science techniques which utilize historical outcomes and deviations in predicted ablation zone characteristics from actual ablation zone characteristics in generating and outputting predicted ablation zones. In an aspect, the algorithm segments the actual ablation zone from the post-procedure scan and compares it to the predicted ablation zone predicted in the preprocedural scan. The 3D volume is used to find the deviation in the 3D space between the plan and the post procedural confirmation.

Although embodiments have been described in detail with reference to the accompanying drawings for the purpose of illustration and description, it is to be understood that the inventive processes and apparatus are not to be construed as limited thereby. It will be apparent to those of ordinary skill in the art that various modifications to the foregoing embodiments may be made without departing from the scope of the disclosure.

1. An ablation system, comprising:
an ablation device configured to navigate to a target within a lung of a patient and ablate the target; and
a computing device configured to:
receive functional respiratory imaging data of the patient;
generate a three dimensional model of the lung of the patient based on the received functional respiratory imaging data of the patient;
predict lung movement based on the received functional respiratory imaging data of the patient;
predict movement of the ablation device relative to the lung and the target during a predetermined duration of ablation; and
predict an ablation zone position and margin relative to the target based on the received functional respiratory imaging data of the patient and the predicted movement of the ablation device relative to the lung and the target during the predetermined duration of ablation.

2. The ablation system of claim 1, wherein the computing device is configured to calculate a path for the ablation device to approach the target that would create a maximum ablation margin based on the received functional respiratory imaging data of the patient.

3. The ablation system of claim 2, wherein the calculated path includes a path through a luminal network and a point on an airway wall to puncture through for placement of the ablation device outside of the luminal network.

4. The ablation system of claim 1, wherein the computing device is configured to calculate a position for final placement of the ablation device relative to the target that would create a maximum ablation margin based on the received functional respiratory imaging data of the patient.

5. The ablation system of claim 4, wherein the calculated position is outside of an airway of the lung.

6. The ablation system of claim 1, wherein the computing device is configured to:
receive post-procedure data corresponding to an actual ablation zone position and margin; and
compare the post-procedural data corresponding to the actual ablation zone position and margin with the predicted ablation zone position and margin to calculate a deviation between the actual ablation zone position and margin with the predicted ablation zone position and margin.

7. The ablation system of claim 6, wherein the computing device is configured to execute a learning algorithm to learn from the calculated deviation for other predictions of ablation zone positions and margins.

8. The ablation system of claim 1, wherein the functional respiratory imaging data includes data corresponding to at least one of blood vessel volume, airway volume, lung volume, airway resistance, internal airflow distribution, ventilation, perfusion, air trapping, aerosol deposition, fissure integrity, fibrosis, or emphysema.

9. An ablation system, comprising:
a computing device configured to:
receive functional respiratory imaging data of a lung of a patient and a target within the lung;
predict lung movement based on the received functional respiratory imaging data of the patient;
predict movement of an ablation device relative to the lung and the target during a predetermined duration of ablation; and
predict an ablation zone position and margin relative to the target based on the received functional respiratory imaging data of the patient and the predicted movement of the ablation device relative to the lung and the target during the predetermined duration of ablation.

10. The ablation system of claim 9, wherein the computing device is configured to generate a three dimensional model of the lung of the patient based on the received functional respiratory imaging data of the patient.

11. The ablation system of claim 9, wherein the computing device is configured to calculate a path for the ablation device to approach the target that would create a maximum ablation margin based on the received functional respiratory imaging data of the patient.

12. The ablation system of claim 11, wherein the calculated path includes a path through a luminal network and a point on an airway wall to puncture through for placement of the ablation device outside of the luminal network.

13. The ablation system of claim 9, wherein the computing device is configured to calculate a position for final placement of the ablation device relative to the target that would create a maximum ablation margin based on the received functional respiratory imaging data of the patient.

14. The ablation system of claim 13, wherein the calculated best position is outside of an airway of the lung.

15. The ablation system of claim 9, wherein the computing device is configured to:
receive post-procedure data corresponding to an actual ablation zone position and margin; and
compare the post-procedural data corresponding to the actual ablation zone position and margin with the predicted ablation zone position and margin to calculate a deviation between the actual ablation zone position and margin with the predicted ablation zone position and margin.

16. The ablation system of claim 15, wherein the computing device is configured to execute a learning algorithm to learn from the calculated deviation for other predictions of ablation zone positions and margins.

17. The ablation system of claim 9, wherein the functional respiratory imaging data includes data corresponding to at least one of blood vessel volume, airway volume, lung volume, airway resistance, internal airflow distribution, ventilation, perfusion, air trapping, aerosol deposition, fissure integrity, fibrosis, or emphysema.

18. An ablation system, comprising:
a computing device configured to:
receive functional respiratory imaging data of a lung of a patient and a target within the lung;
predict an ablation zone position and margin relative to the target based on the received functional respiratory imaging data of the patient;
receive post-procedure data corresponding to an actual ablation zone position and margin; and
compare the post-procedural data corresponding to the actual ablation zone position and margin with the predicted ablation zone position and margin to calculate a deviation between the actual ablation zone position and margin with the predicted ablation zone position and margin.

19. The ablation system of claim 18, wherein the computing device is configured to execute a learning algorithm to learn from the calculated deviation for other predictions of ablation zone positions and margins.

20. The ablation system of claim 18, wherein the functional respiratory imaging data includes data corresponding to at least one of blood vessel volume, airway volume, lung volume, airway resistance, internal airflow distribution, ventilation, perfusion, air trapping, aerosol deposition, fissure integrity, fibrosis, or emphysema.
